(19) United States
(12) Patent Application Publication
(10) Pub. No.: US 2004/0242702 A1
(43) Pub. Date: Dec. 2, 2004

(54) Glutaraldehyde composition

(76) Inventor: Antonietta Pamela Martin, Parkhurst (ZA)

Correspondence Address: Maxwell J Petersen, Pauley Petersen & Erickson, 2800 West Higgins Road, Suite 365, Hoffman Estates, IL, 60195, US

(21) Appl. No.: 10/485,070

(22) PCT Filed: Jul. 26, 2002
(86) PCT No.: PCT/IB02/02913

(30) Foreign Application Priority Data
Jul. 27, 2001 (NZ) 513213

(51) Int. Cl. A61K 31/11

(52) U.S. Cl. 514/693

(57) ABSTRACT

This invention provides stable aqueous glutaraldehyde solution comprising 0.005% to 45% m/v solution of glutaraldehyde, an alcohol exthoxylate non-ionic surfactant, sodium acetate trihydrate and sufficient of a pH modifier to bring the pH of the solution to 6.0 to 8.5. In particular, the invention relates to stable concentrate solutions comprising 10% to 45% m/v glutaraldehyde. These stable solutions can be stored for a period of at least six months without the glutaraldehyde polymerising or the pH dropping below 5.0.

### BACKGROUND OF THE INVENTION

THIS invention relates to a glutaraldehyde composition.

Glutaraldehyde in solution is well known as either a cleaning, disinfecting or sterilizing agent. It has been shown to be a powerful external bactericidal, fungicidal and virucidal agent. For glutaraldehyde to be effective as such, however, the pH of the glutaraldehyde solution must be in the range of 7 to 8.5. It is difficult to maintain glutaraldehyde solutions at this pH as they are unstable.

South African patent no. 93/0662 discloses an aqueous glutaraldehyde solution which comprises 4 to 6% m/v of glutaraldehyde, 19 to 21% m/v nonylphenyl ethoxylate, sufficient of a pH modifier to bring the pH of the solution to 6 to 7.5 and sodium acetate trihydrate in an amount that, with the nonylphenyl ethoxylate, serves to buffer the solution at the specified pH range. Such solutions have been found to be stable for a period of up to six months.

It is an object of this Invention to provide a new aqueous glutaraldehyde solution.

### SUMMARY OF THE INVENTION

According to the invention a stable aqueous glutaraldehyde solution comprises:

a 0.005% to 45% m/v solution of glutaraldehyde (OCH(CH₂)₃CHO);

an alcohol ethoxylate non-ionic surfactant;

sodium acetate trihydrate (NaC₂H₃O₂); and

sufficient amount of a pH modifier to bring the pH of the solution to 6.0 to 8.5.

By “stable” in relation to the concentrate of the invention, it is meant that the concentrate can be stored for a period of at least six months without the glutaraldehyde polymerising or the pH dropping below 5.0.

A concentrate solution typically comprises 10% to 45% m/v glutaraldehyde and as much sodium acetate trihydrate as is required to buffer the pH of the solution at 6.0 to 8.5, typically about 0.05 to 0.5% m/v of sodium acetate trihydrate.

Preferably, the solution contains sufficient pH modifier to bring its pH to 7.5.

The alcohol ethoxylate non-ionic surfactant, for example Tergitol-15S9, is typically at a pH of 8.0 to 9.0, and together with the sodium acetate trihydrate, functions as a buffer to maintain the aqueous solution of the invention at the pH of 6 to 8.5. Typically, the alcohol ethoxylate non-ionic surfactant makes up from 0.6 to 25% m/v of the solution. This surfactant is chemically bound to the glutaraldehyde and stabilizes the glutaraldehyde in a more effective linear configuration.

The pH modifier may be a base, for example it may be a dilute aqueous solution of sodium hydroxide (NaOH). It is preferably a 1M aqueous NaOH solution.

The solution may also contain a quaternary ammonium compound (QAC), preferably a twin chain quarternary ammonium compound. Advantageously, the twin chain quaternary compound makes up from 0.1% to 15% m/v of the solution.

A concentrate solution of the invention may be diluted to produce an aqueous sterilising, cleaning, disinfecting, antiseptic, subcutaneous injectable or preservative end use composition, having pH of 5 to 8.5.

Typically, these compositions are formed by diluting a concentrate solution of the invention with sterile or potable water having a pH of 6 to 8.5 to provide a composition of reduced glutaraldehyde concentration.

Preferably, the concentration of glutaraldehyde in these diluted end use compositions is in the range of 0.005% to 5% m/v.

According to another aspect of the invention there is provided a method of producing any one of the stable aqueous glutaraldehyde solutions mentioned above, the method including the steps of:

a) heating water to a temperature between 45° C. to 50° C.;

b) adding an alcohol ethoxylate non-ionic surfactant to the water while maintaining the temperature of the solution so formed between 45° C. to 50° C.;

c) adding a glutaraldehyde solution to the solution;

d) maintaining the temperature of the solution at 45° C. to 50° C. for a period of 15 to 30 minutes to allow the glutaraldehyde to cross-link with the alcohol ethoxylate non-ionic surfactant;

e) optionally adding other active ingredients to the solution and decreasing the temperature of the solution by adding water to the solution;

f) adjusting the pH of the solution to 7.5 to 8.5 using a pH modifier; and

g) adding sodium acetate trihydrate to the solution to buffer the solution to a pH of 6.0 to 8.5.

The invention also covers a stabilised glutaraldehyde composition having an ESI-HPLC-MS spectrum where the spectral detail is populated in the high mass range (from just below 500 m/z to approximately 900 m/z) of the spectrum, typically a spectral detail substantially the same as that shown in FIG. 4 having a parabolic profile with a homologous series 44 m/z (—CH₂—CH₂—).

### BRIEF DESCRIPTION OF THE DRAWINGS

FIG. 1 is an ESI-HPLC-MS spectrum of glutaraldehyde for use in a stabilised glutaraldehyde composition of the invention;

FIG. 2 is an ESI-HPLC-MS spectrum of an alcohol ethoxylated non-ionic surfactant for use in a stabilised glutaraldehyde composition of the invention;

FIG. 3 is an ESI-HPLC-MS spectrum of buffer chemicals (including sodium acetate trihydrate and sodium hydroxide) for use in a stabilised glutaraldehyde composition of the invention; and

FIG. 4 is a an ESI-HPLC-MS spectrum of a stabilised glutaraldehyde composition of the invention.

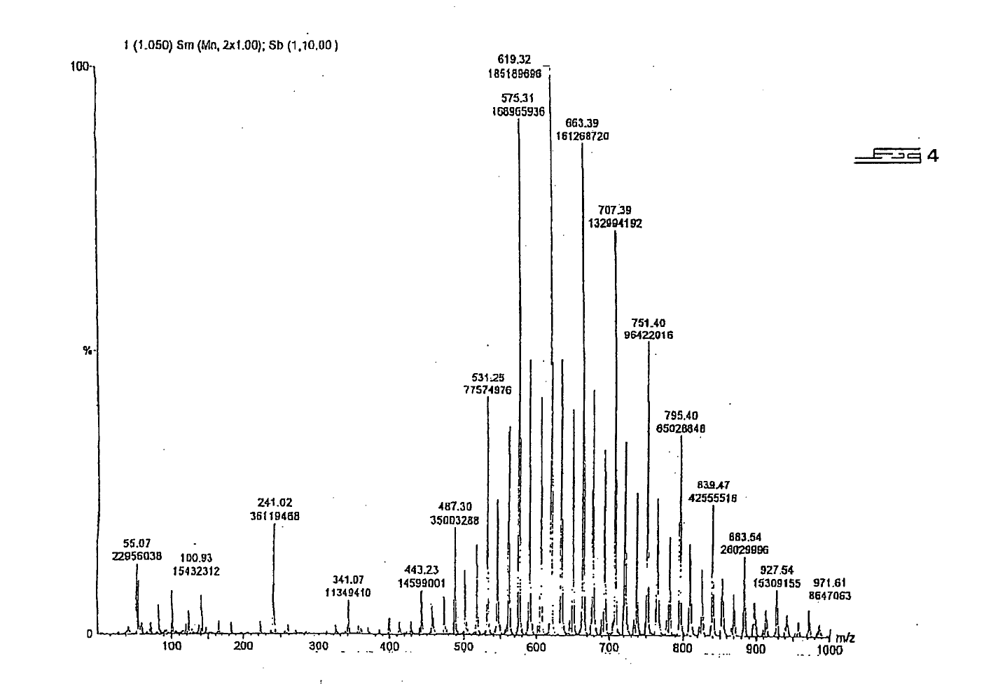

### DESCRIPTION OF EMBODIMENTS

The inventor has now developed a new stable concentrate glutaraldehyde solution using a new non-ionic surfactant, namely an alcohol ethoxylate non-ionic surfactant. The inventor has also developed a new concentrate glutaraldehyde solution, the solution having a higher concentration of glutaraldehyde, i.e. the concentrate solution comprises from 10% to 45% m/v of an aqueous solution of glutaraldehyde. Typical concentrate solutions contain 10%, 20%, 30% and 45% m/v glutaraldehyde, depending on the application of the solution.

A concentrate solution contains 0.6 to 0.25% m/v the alcohol ethoxylate non-ionic surfactant (with a pH of 8.0 to 9.0), sodium acetate trihydrate to buffer the pH of the aqueous solution at about 6.0 to 8.5 and sufficient amount of a pH modifier (such as sodium hydroxide) to bring the pH of the aqueous solution up to 6.0 to 8.5, preferably 7.5. Usually the concentrate solution will include about 0.05%-0.5% m/v sodium acetate trihydrate. In addition, the aqueous solution may contain a twin chain quaternary ammonium compound which has increased fungicidal activity of the solution and which also increases the foaming ability of the solution. The increased foaming ability provides a larger surface contact time on application of the product when sprayed onto large surfaces and thus increases the overall efficacy of the product. A twin chain quaternary ammonium compound is used in preference to a single chain of quaternary ammonium compound. The twin chain quaternary ammonium compound has greater steric hinderance to protect the nitrogen atom of the quaternary ammonium compound, making it more stable than glutaraldehyde and a single chain quaternary ammonium compound.

A twin chain quaternary ammonium compound has the formula R₂—N(CH₃)₂Cl⁻ where R represents an alkyl straight chain. One preferred example is 1-decanamminium, N-decyl-N,N-dimethyl-chloride.

A glutaraldehyde composition according to the invention is prepared by heating water up to a temperature between 45° C. to 50° C. Sufficient alcohol ethoxylate derivative is added to provide a concentrate containing from 0.6 to 25% m/v of alcohol ethoxylate derivative, while maintaining the temperature of the solution at 45° C. to 50° C. Next, a required amount of glutaraldehyde solution is added to provide a concentrate having a glutaraldehyde concentration of 10 to 45% m/v, while keeping the temperature at 45° C. to 50° C. The glutaraldehyde and detergent are allowed to react, while maintaining the temperature at 45° C. to 50° C. for a period of 15 to 30 minutes. During this time, the glutaraldehyde is cross-linked with the surfactant to create the following molecule:

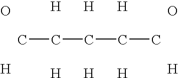

Where R equals a 15 carbon long alcohol ethoxylated non-ionic surfactant. Cold water is then added to bring the solution up to 75% of the total volume of the concentrate solution.

Thereafter, all, some or one of the following ingredients may be added, depending on the application:

a) Sufficient amount of a twin chain quaternary ammonium compound to provide a 0.1%-15% m/v concentration in the concentrate.

b) Sufficient amount of a degreasing additive, for example a glycolic solvent to provide a concentration of 0.5%-2% m/v in the concentrate.

c) Sufficient amount of a glycolic acid to provide a 0.5%-30% m/v concentration in the concentrate.

d) Sufficient amount of sodium lauryl ethyl sulphate to provide a concentration of 5%-20% m/v of the concentrate.

e) Sufficient amount of water soluble silicon products to form a 0.01%-0.2% m/v of the concentrate and to act as a defoamer in certain applications.

After all, or some, or one of the above ingredients is added, the solution is brought up to its final volume with water. Thereafter, the pH of the solution is adjusted to 7.5 to 8.5 with a pH modifier such as sodium hydroxide (typically a 1 M aqueous solution). Thereafter, sodium acetate trihydrate is added to buffer the product at a pH of 6.0 to 8.5.

An anti-corrosive inhibitor such as sodium nitrate may then be added, and dyes may be added for aesthetic purposes, and liquid fragrances (that are non-nitrogen based) may also be added.

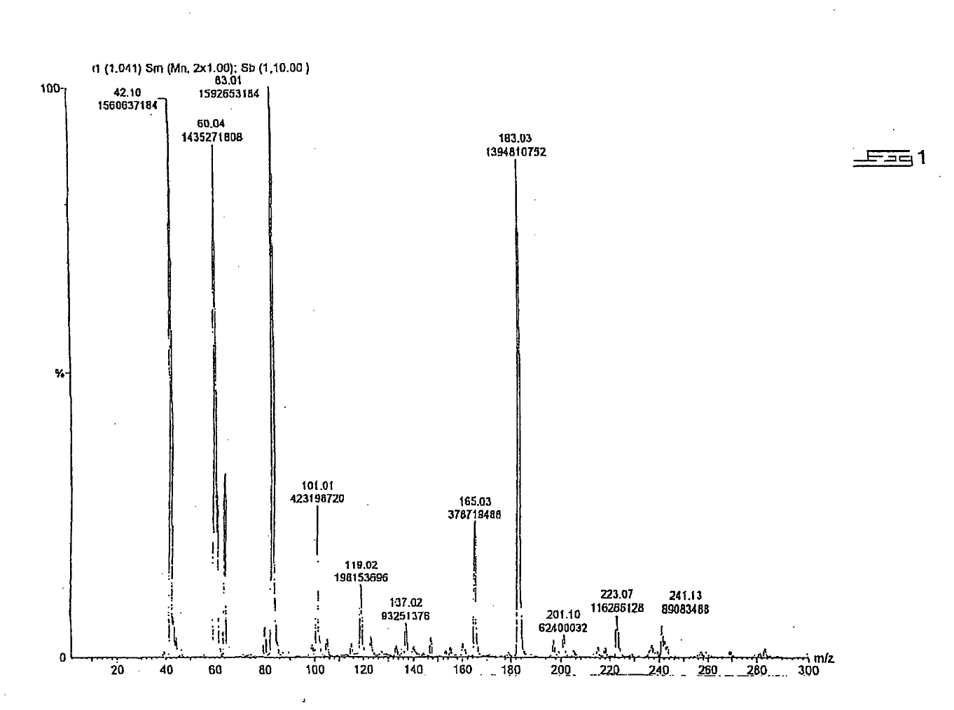

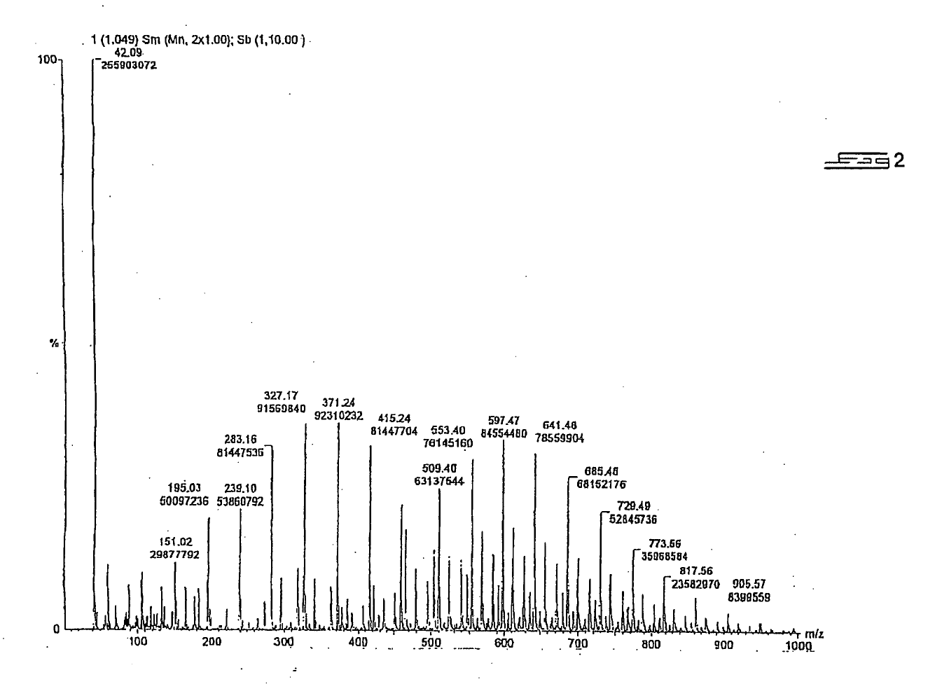

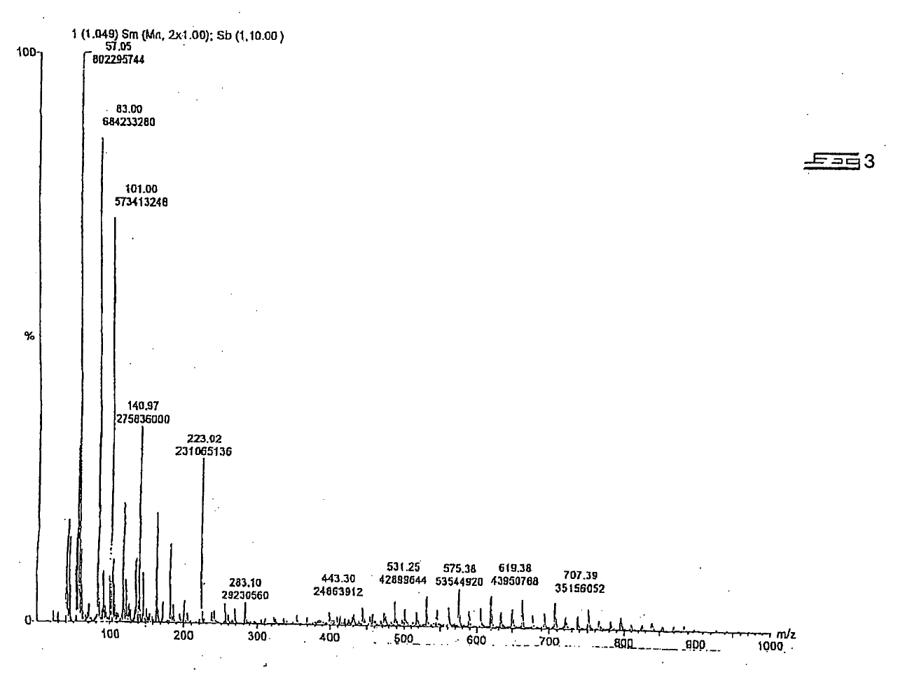

We refer now to the Figures. It will be seen that the ESI-HPLC-MS spectrum of the stabilised glutaraldehyde composition of the invention is very different from the starting materials shown in FIGS. 1-3. In particular, it will be noted that the spectrum for the alcohol ethoxylated non-ionic surfactant has a relatively flat profile with a homogeneous series of 44 m/z (—CH₂CH₂—). It would seem that the spectral detail is populated relatively uniformly across the mass range of the spectrum (the low mass range as well as the high mass range) with no region dominating in particular. While the spectrum for the stabilised glutaraldehyde composition of the invention (i.e. the glutaraldehyde cross-linked with the alcohol ethoxylate non-ionic surfactant) has the appearance of a parabolic profile with a homologous series of 44 m/z (—CH₂—CH₂—). The spectral detail is populated at the high mass range (from just below 500 m/z to approximately 900 m/z) of the spectrum with relatively little detail in the low mass region (except for the 241 m/z ion). The highest ions in the given mass spectrum are 575.31 m/z, 619.32 m/z and 663.39 m/z.

The glutaraldehyde solutions described above have a shelf life of a minimum of six months.

In use, the concentrate solution is diluted to provide end use compositions being in the range of 0.001% to 5% m/v glutaraldehyde. When diluted, the end use compositions will typically have a pH of 6 to 8.5 which is the pH at which glutaraldehyde is optimally activated. At this neutral, or close to neutral pH, the solutions are non-corrosive and non-irritant and hence user friendly. Further, the concentrate glutaraldehyde solutions are compatible with products that may be used in end-use compositions, such as: defoamers (e.g. water-soluble synthetic silicon type products); degreasers (e.g glycols); surfactants (e.g. sodium lauryl ethyl sulphate, triethalamines and cocoamides); thickeners (e.g. cellulose hydroxyls and starch thickeners), pH activators (e.g. citric acid); pesticides (e.g. permethrine) and other products that do not contain non-steric hindered nitrogen elements.

The product can be used in many applications, including in domestic cleaning products, personal hygiene products, as a water preservative, in sewage water purification, in pharmaceutical products as an effective biocide, in preventative medication including disinfecting of needles or washing needles, stabs or pricks etc. It is also useful as a machine cleaner in combination with other chemicals, such as glycolic acid, as an animal skin care washing or dipping products or for the cleaning of food products. The product is also compatible with degreasing chemicals, for example glycols.

End-use compositions made by diluting a concentrate composition according to the invention are set out below:

| | Percent(%) by Weight |
|---|---|
| End-Use Composition | Active Glutaraldehyde |
| Medical Instrument Sterilent | 2.0%-3.2% |
| Domestic Household Cleaner | 0.3%-0.5% |
| Toilet Cleaner | 1%-5% |
| Waste Water Treatment | 30%-45% |
| | concentrates which |
| | are diluted to |
| | 0.001%-0.025% |
| | when applied to |
| | waste water |
| Dairy Machine Cleaner | 0.1%-6% |
| Skin Cleansers and Creams | 0.3%-0.5% |
| Injectables and Skin | 0.005%-0.3% |
| Preventative Application | |
| Abattoir Cleaners and | 0.1%-5% |
| Degreasers for all Surfaces | |
| Wall and Surface Cleaners | 0.005%-1% |
| for the Poultry Industry | |
| Surface Disinfectants for | 0.025%-1% |
| Food Processing Industries | |
| Cell Culturing, Planting, | 0.05%-0.3% |
| Seedling Growing and | |
| Watering Systems | |
| Pesticide and Biocide | 0.025%-1% |
| Wipes and Swabs | 0.1%-0.3% |

### EXAMPLES

### Example 1

### 10% m/v Concentrate Solution of Glutaraldehyde

The ingredients of a 10% m/v low foam concentrate solution of glutaraldehyde according to the invention is set out below:

a) 200-226 kg Ucarcide 250 50% w/w glutaraldehyde;

b) 6-22 kg Tergitol 15.S9;

c) 6-50 kg twin chain quaternary ammonium compound (1-decanamminium, N-decyl-N,N-dimethyl-chloride);

d) 2.5 kg-10 kg sodium acetate trihydrate;

e) sufficient sodium hydroxide to bring the pH of the solution to 7.5; and

f) 1-2 kg Dowanol DPM glycol (degreaser).

The ingredients of a 1000 l 10% m/v high foam concentrate solution of glutaraldehyde is set out below:

a) 200-226 kg Ucarcide 250 50% w/w glutaraldehyde;

b) 100-150 g Tergitol 15.S.9;

c) 6-50 kg twin chain quaternary ammonium compound (1-decanamminium, N-decyl-N,N-dimethyl-chloride);

d) 2.5 kg-10 kg sodium acetate trihydrate;

e) sufficient sodium hydroxide to bring the pH of the solution to 7.5; and

f) 1-2 kg Dowanol DPM gycol (degreaser).

### Example 2

### 20% m/v Glutaraldehyde Concentrate Composition

The ingredients of a 1000 t 20% m/v glutaraldehyde solution of the invention is as set out below:

a) 400-452 kg Ucarcide 250 50% w/w glutaraldehyde;

b) 6-22 kg Tergitol 15.S.9;

c) 6-50 kg twin chain quaternary ammonium compound (1-decanamminium, N-decyl-N,N-dimethyl-chloride);

d) 5 kg-15 kg sodium acetate trihydrate;

e) sufficient sodium hydroxide to bring the pH of the solution to 7.5; and

f) 1-2 kg Dowanol DPM glycol (degreaser).

### Example 3

### 30% m/v Glutaraldehyde Concentrate Composition

The ingredients of a 1000 l 30% M/v glutaraldehyde solution of the invention is as set out below:

a) 600-678 kg Ucarcide 250 50% w/w glutaraldehyde;

b) 6-22 kg Tergitol 15.S.9;

c) 6-50 kg twin chain quaternary ammonium compound (1-decanamminium, N-decyl-N,N-dimethyl-chloride);

d) 10 kg-20 kg sodium acetate trihydrate;

e) sufficient sodium hydroxide to bring the pH of the solution to 7.5; and

f) 1-2 kg Dowanol DPM glycol (degreaser).

### Example 4

### 45% m/v Glutaraldehyde Concentrate Composition

The ingredients of a 1000 l 45% m/v glutaraldehyde solution of the invention is as set out below:

a) 900 kg Ucarcide 250 50% w/w glutaraldehyde;

b) 6-22 kg Tergitol 15.S.9;

c) 6-50 kg twin chain quaternary ammonium compound (1-decanamminium, N-decyl-N,N-dimethyl-chloride);

d) 10 kg-20 kg sodium acetate trihydrate;

e) sufficient sodium hydroxide to bring the pH of the solution to 7.5; and

f) 1-2 kg Dowanol DPM glycol (degreaser).

1. A stable aqueous glutaraldehyde solution comprising:
a 0.005% to 45% m/v solution of glutaraldehyde (OCH(CH₂)₃ CHO);
an alcohol ethoxylate non-ionic surfactant;
sodium acetate trihydrate (NaC₂H₃O₂); and
sufficient amount of a pH modifier to bring the pH of the solution to 6.0 to 8.5.

2. A concentrate solution according to claim 1 comprising 10% to 45% m/v glutaraldehyde.

3. A solution according to claim 1 comprising as much sodium acetate trihydrate as is required to buffer the pH of the solution at 6.0 to 8.5.

4. A solution according to claim 3 comprising about 0.05 to 0.5% m/v sodium acetate trihydrate.

5. A solution according to claim 1 comprising sufficient pH modifier to bring its pH to 7.5.

6. A solution according to any one of the preceding claims claim 1 wherein the alcohol ethoxylate non-ionic surfactant has a pH of 8.0 to 9.0 and, together with the sodium acetate trihydrate, functions as a buffer to maintain the aqueous solution of the invention at the pH of 6.0 to 8.5.

7. A solution according to claim 6 wherein the alcohol ethoxylate non-ionic surfactant makes up from 0.6 to 25% m/v of the solution.

8. A solution according to claim 7 wherein the alcohol ethoxylate non-ionic surfactant comprises Tergitol-15 S9.

9. A solution according to claim 1 wherein the pH modifier is comprises a dilute aqueous solution of sodium hydroxide (NaOH).

10. A solution according to claim 9 wherein the pH modifier comprises a 1M aqueous NaOH solution.

11. A solution according to claim 1 further comprising a quaternary ammonium compound.

12. A solution according to claim 11 wherein the quaternary ammonium compound is a twin chain quaternary ammonium compound.

13. A solution according to claim 12 wherein the twin chain quaternary ammonium compound makes up from 0.1% to 15% m/v of the solution.

14. A method of producing a stable aqueous glutaraldehyde solutions comprising the steps of:
a) heating water to a temperature between 45° C. to 50° C.;
b) adding an alcohol ethoxylate non-ionic surfactant to the water to form a solution, while maintaining the temperature of the solution so formed between 45° C. to 50° C.;
c) adding a glutaraldehyde solution to the solution;
d) maintaining the temperature of the solution at 45° C. to 50° C. for a period of 15 to 30 minutes to allow the glutaraldehyde to cross-link with the non-ionic alcohol ethoxylate surfactant;
e) optionally adding other active ingredients to the solution and decreasing the temperatures of the solution by adding water to the solution;
f) adjusting the pH of the solution to 7.5 to 8.5 using a pH modifier; and
g) adding sodium acetate trihydrate to the solution to buffer the solution to a pH of 6.0 to 8.5.

15. A stabilised glutaraldehyde composition having an ESI-HPLC-MS spectrum where the spectral detail is populated in the high mass range (from just below 500 m/z to approximately 900 m/z) of the spectrum.

16. (Canceled)

17. A solution according to claim 2 comprising as much sodium acetate trihydrate as is required to buffer the pH of the solution at 6.0 to 8.5.

18. A solution according to claim 17 comprising about 0.05 to 0.5% m/v sodium acetate trihydrate.

19. A solution according to claim 18 comprising sufficient pH modifier to bring its pH to 7.5.

20. A solution according to claim 18 wherein the alcohol ethoxylate non-ionic surfactant makes up from 0.6 to 25% m/v of the solution.

21. A solution according to claim 18 wherein the pH modifier comprises a dilute aqueous solution of sodium hydroxide.

22. A solution according to claim 18 further comprising a quaternary ammonium compound.
